(19) United States
(12) Patent Application Publication
(10) Pub. No.: US 2026/0234527 A1
(43) Pub. Date: Aug. 13, 2026

(54) LOCKING CAROUSEL WITH LOCATING FEATURES

(71) Applicant: RAPID MICRO BIOSYSTEMS, INC., Lowell, MA (US)

(72) Inventor: Peter TIA, Winchester, MA (US)

(21) Appl. No.: 19/053,000

(22) Filed: Feb. 13, 2025

(51) Int. Cl. C12M 3/00; C12M 1/00; C12M 1/22; C12M 1/36

(52) U.S. Cl. CPC C12M 23/48; C12M 23/10; C12M 23/50; C12M 41/48

(57) ABSTRACT

An alignment system is described for aligning a carousel of a microbial detection and/or enumeration system to an indexer configured to rotate the carousel into predetermined locations. The alignment system may include an indexer-side positioning element and a carousel-side positioning element. The positioning elements may include interfacing features that allow the carousel to be automatically aligned into a predetermined configuration with respect to the indexer as the carousel is lowered onto the indexer. For example, the indexer-side positioning element may include a spindle that is received in an opening of the carousel-side positioning element. Ball bearings on either element may interface with grooves on the other element to guide the elements into position with respect to each other. Optionally, magnets one or both elements may be used to releasably secure the elements together.

### BACKGROUND

Many industries require the determination of the number or presence of microbes in a sample, often referred to as microbe enumeration or detection. One method of determining the number of microbes in a sample (or detecting the presence of microbes) involves exposing a filtration membrane to a sample to capture microbes in the sample on the membrane, culturing the microbes captured on the membrane, and optionally counting the number of colonies that grow during culturing.

The membrane may be provided inside a cassette to protect the microbe culture during incubation and/or analysis. The cassette may also facilitate automated processing by holding and organizing samples as they move through various stages of processing. Examples of automated sample processing systems include the GROWTH DIRECT™ and GROWTH DIRECT™ Rapid Sterility systems from RAPID MICRO BIOSYSTEMS™ of Lowell, Massachusetts.

An operator may load the cassettes into a carousel that allows multiple cassettes to be analyzed in an automated sample analysis system. The system may retrieve the cassettes from the carousel and perform an analysis on the samples in the cassette. During some types of automated sample analysis, illuminated samples are caused to fluoresce without destroying the samples. By combining digital imagining technology with sophisticated software algorithms, these systems can detect and count the autofluorescence of growing microbes.

In order to facilitate automated processing, it is important that the carousel be rotatable into predictable positions, and that the current position of the carousel be determinable. If the carousel does not rotate into the intended position with some degree of precision, then the specific sample to be retrieved from the carousel may not be in the correct location and an incorrect sample may be retrieved. Moreover, there can sometimes be problems in these systems when an object strikes the carousel or there is some other external disturbance, which may cause the carousel to move or rotate such that the cassettes are displaced from their intended alignment.

### BRIEF SUMMARY

This application relates to an alignment system for aligning a carousel with respect to an indexer in a microbial detection and/or enumeration system. The carousel is configured to hold one or more cassettes and to be rotated into predefined positions by the indexer. Also described are methods for manufacturing and using the alignment system.

The alignment system may include a carousel-side positioning element configured to be affixed to (or formed integrally with) to the carousel and an indexer-side positioning element configured to be affixed to (or formed integrally with) the indexer. The carousel-side positioning element may be configured to interface with the indexer-side positioning element to guide the carousel into a predetermined position when the carousel is deployed on the carousel.

The indexer-side positioning element may include an indexer-side plate and a spindle, the spindle sized and shaped to be received in a corresponding opening of the carousel-side positioning element. The spindle may include one or more grooves and the opening of the carousel-side positioning element includes one or more protrusions extending into the opening, where the protrusions are sized, shaped, and positioned in the opening to interface with the grooves of the spindle as the carousel-side positioning element is lowered onto the indexer-side positioning element. The spindle may taper from a distal end to a proximal end to guide the carousel-side positioning element into place as the carousel-side positioning element is lowered onto the spindle.

The indexer-side plate may include one or more ball bearings sized positioned to be received in corresponding grooves in the carousel-side positioning element to position the indexer-side positioning element with respect to the carousel-side positioning element. Alternatively, the carousel-side plate may include one or more ball bearings sized positioned to be received in corresponding grooves in the indexer-side positioning element to position the indexer-side positioning element with respect to the carousel-side positioning element. Still further, the ball bearings and grooves may be combined so that both plates include both ball bearings and grooves.

The indexer-side positioning element may include one or more magnets and the carousel-side positioning element includes one or more corresponding magnets configured to attract the one or more magnets of the indexer-side positioning element. Alternatively, the indexer-side positioning element or the carousel-side positioning element may include one or more magnets, and the other of the indexer-side positioning element and the carousel-side positioning element includes one or more ferrous surfaces configured to attract the one or more magnets.

The carousel-side positioning element may include a carousel-side plate configured to be attached to a base of the carousel, where the carousel-side plate includes an opening configured to receive a spindle of the indexer-side positioning element.

The alignment system may also include one or more fasteners configured to secure the indexer-side positioning element to the indexer, the carousel-side positioning element to the carousel, or the indexer-side positioning element to the carousel through the carousel-side positioning element.

In some examples, the indexer and the carousel may be provided as separate elements that can be combined into the alignment system. For example, the indexer may include an indexer body housing a motor, an indexer-side plate configured to be rotated by the motor, and a spindle configured to receive the carousel-side positioning element. The spindle may include one or more grooves configured to position the carousel with respect to the indexer as the carousel-side plate is lowered onto the spindle.

In another example, a carousel may include one or more cassette columns, a carousel base supporting the one or more cassette columns, and a carousel-side positioning element configured to position the carousel with respect to the indexer. The carousel-side positioning element may include an opening configured to receive a spindle of the indexer, the opening including one or more protrusions corresponding to grooves in the spindle.

A method is also described for manufacturing a system for positioning a carousel with respect to an indexer configured to rotate the carousel into predefined positions. The method may include forming an indexer spindle on an indexer-side plate, the indexer spindle extending from a proximal end closest to the indexer-side plate to a distal end and tapering from a relatively large diameter at the proximal end to a relatively small diameter at the distal end. One or more grooves may be formed in the indexer spindle. A carousel-side plate may be accessed. An opening may be formed in the carousel-side plate, where the forming includes shaping one or more protrusions in the opening to correspond to the one or more grooves of the indexer spindle.

If magnets are to be used to secure the carousel to the indexer, the method may include adding magnets to one or more of the indexer-side plate or the carousel-side plate.

The method may also include securing one or more ball bearings in the indexer-side plate and forming one or more grooves in the carousel-side plate configured to receive the ball bearings to guide the carousel-side plate onto the indexer-side plate. Alternatively, the method may also include securing one or more ball bearings in the carousel-side plate and forming one or more grooves in the indexer-side plate configured to receive the ball bearings and to guide the carousel-side plate onto the indexer-side plate.

The method may also include fixing the carousel-side plate to the carousel using one or more carousel-side fasteners, and fixing the indexer-side plate to the indexer using one or more indexer-side fasteners.

In one aspect, a method of using a system for positioning a carousel with respect to an indexer configured to rotate the carousel into predefined positions, the method includes attaching a carousel-side positioning element to the carousel, the carousel-side positioning element includes an opening, attaching an indexer-side positioning element to the indexer, the indexer-side positioning element includes a spindle configured to be received in the opening of the carousel-side positioning element, and dropping the carousel-side positioning element onto the indexer-side positioning element, where dropping the carousel-side positioning element causes one or more protrusions in the opening of the carousel-side positioning element to be received in one or more corresponding grooves on the spindle to automatically align the carousel with the indexer.

The method may also include further includes aligning one or more magnets on the carousel-side positioning element to one or more corresponding magnets on the indexer-side positioning element to secure the carousel to the indexer.

The method may also include further includes aligning one or more ball bearings on one of the carousel-side positioning element or the indexer-side positioning element to one or more grooves of the other of the carousel-side positioning element or the indexer-side positioning element.

Other technical features may be readily apparent to one skilled in the art from the following figures, descriptions, and claims.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 depicts an exemplary microbe detection/enumeration system.

FIG. 2A depicts an exemplary carousel holding stacks of cassettes in a microbe detection/enumeration system.

FIG. 2B depicts the carousel of FIG. 2A from another perspective.

FIG. 3 illustrates an example carousel being lowered onto an example indexer.

FIG. 4 depicts the carousel and indexer of FIG. 3 after the carousel has been engaged with the indexer.

FIG. 5 provides a close-up perspective view of an example indexer.

FIG. 6A depicts an example of a carousel-side positioning element being engaged with an indexer-side positioning element.

FIG. 6B depicts an example of a carousel-side positioning element being engaged with an indexer-side positioning element.

FIG. 6C depicts an example of a carousel-side positioning element being engaged with an indexer-side positioning element.

FIG. 6D depicts an example of a carousel-side positioning element being engaged with an indexer-side positioning element.

FIG. 7 depicts an alternative example of a carousel-side positioning element having ball bearings.

FIG. 8 depicts the alternative example from FIG. 7 from a different perspective.

FIG. 9 is a top-down view of a carousel-side positioning element positioned on top of an indexer-side positioning element.

FIG. 10 depicts an example of an indexer having grooves configured to receive the ball bearings in the alternative carousel-side positioning element from FIG. 7 and FIG. 8.

FIG. 11 is a side-view of an example alignment system showing the locations of through-holes for receiving fasteners.

FIG. 12 is a side view of an example alignment system showing the location of one of the ball bearings and corresponding groove.

FIG. 13 is a flow chart depicting a method for manufacturing an example alignment system.

FIG. 14 is a flow chart depicting a method for deploying, using, and taking apart an example alignment system.

### DETAILED DESCRIPTION

Exemplary embodiments relate to a rotary carousel that attaches a rotary indexing mechanism with precise locating features. Some examples include magnets on the rotary indexing mechanism and corresponding magnets on the carousel to secure the components in place. The magnets may be selected so as to be sufficiently strong to secure the components in place during typical use, while being able to break away if sufficient force is applied. This allows the carousel to be lifted out of position, such as when an object crashes into the carousel from below. Once the obstructing object is lowered, the carousel will lower back into position and secure in place due to the force of the magnets.

In some examples, ball bearings on the carousel meet with a V-shaped groove on the indexing mechanism for precise alignment of the system. Optionally, a center spindle may include grooves to align the carousel with the indexer. The center spindle may be sloped to guide the carousel into position, even if the carousel is improperly placed.

The height of the carousel may be selected to be sufficiently tall that a user can install the carousel even if the user is unable to see the spindle.

The described embodiments may find application in several different areas of microbial detection and enumeration, such as sterility testing, environmental monitoring, and bioburden testing.

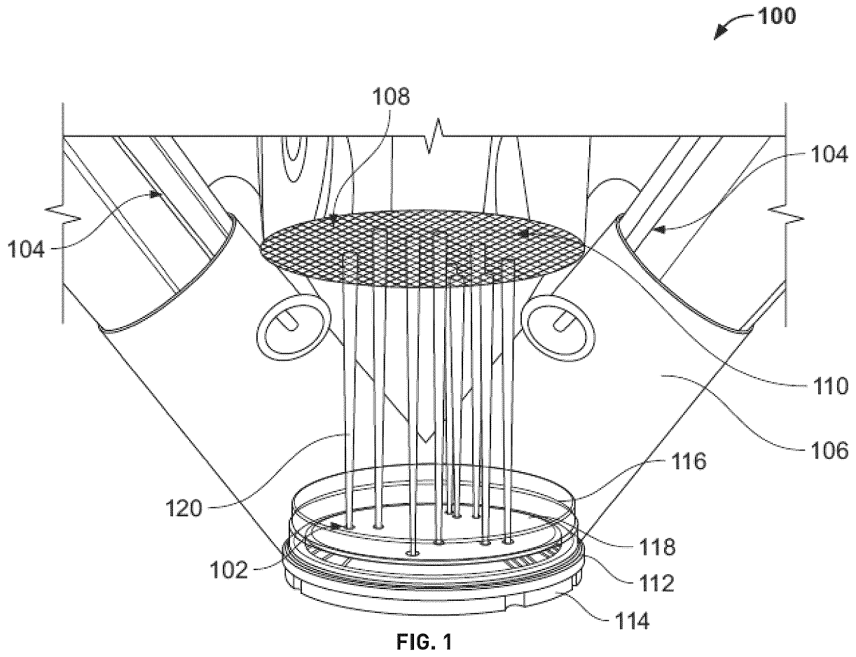

FIG. 1 illustrates an example of an automated sample analysis system 100, according to embodiments described herein. The analysis system 100 comprises microorganism colonies 102, Lamps 104 emitting light 106, a charge-couple device (CCD) chip 108, multiple photosensitive pixels 110, a cassette 112, a cassette base 114, a cassette lid 116, a growth media 118, and microorganism fluorescence 120.

In some embodiments, the CCD chip 108 comprises photosensitive pixels 110 to capture light emitted from the microorganisms 102.

In some embodiments, each cassette 112 comprises a cassette base 114 and a cassette lid 116. In some embodiments, the cassette base 114 contains growth media 118. In some embodiments, the growth media 118 contains microorganism colonies 102. In some embodiments, the microorganism colonies 102 in the growth media 118 are exposed to Lamp light 106 produced by the Lamps 104. In some embodiments, the microorganisms in the microorganism colonies 102 produces microorganism fluorescence 120. In some embodiments, the microorganism fluorescence 120 are captured by the photosensitive pixels 110 of the CCD chip 108.

In some embodiments, the analysis system 100 comprises one or more lamps 104. In some embodiments, the at least one lamp is a light emitting diode (LED) lamp, an incandescent lamp, a fluorescent lamp, a halogen lamp, a high-intensity discharge (HID) lamp, a neon lamp, a laser lamp, a visible light lamp, an ultraviolet (UV) lamp, an infrared (IR) lamp, or any other type of light source capable of producing light. In some embodiments, the sun produces light for the analysis system 100.

In some embodiments, the light 106 produced by the Lamps 104 is incandescent lamp light, fluorescent lamp light, halogen lamp light, high-intensity discharge (HID) lamp light, neon lamp light, laser lamp light, visible light, ultraviolet (UV) light, infrared (IR) light, or any other type of light.

In some embodiments, the analysis system 100 comprises at least one cassette 112. In some embodiments, the growth media 118 comprises one type of microorganism colony 102. In some embodiments, the growth media 118 comprises more than one type of microorganism colony 102. The microorganism colonies 102 may, when exposed to the light 106, produce microorganism fluorescence 120. In some embodiments, different types of microorganism colonies 102 may be distinguished by the different types of microorganism fluorescence 120 that each produces.

In some embodiments, the analysis system 100 comprises one or more CCD chips 108. The CCD chips 108 may include one or more photosensitive pixels 110. The photosensitive pixels 110 may convert the microorganism fluorescence 120 into electrical signals. In some embodiments, the electrical signals are interpreted by logic on the analysis system 100 to indicate the number of microorganism colonies 102 in the growth media 118. In some embodiments, an imaging device other than a CCD chip 108 may be used to capture the microorganism fluorescence 120. In some embodiments, the imaging device is a complementary metal-oxide-semiconductor (CMOS) image sensor, a specific type of CMOS image sensor (such as an active pixel sensor CMOS), a scientific CMOS (sCMOS) image sensor, an indium gallium arsenide (InGaAs) image sensor, other types of CCD chips (such as an electron-multiplying CCD chip), or any other type of sensor capable for imaging.

In some embodiments, the cassette lid 116 of the cassette 112 is substantially transparent. The cassette lid 116 may be considered substantially transparent when light emitted by a selected light source is capable of passing from one side of the cassette lid 116 to another such that the light remains perceptible by a sensor after passing through the cassette lid 116 from side to side. A substantially transparent cassette lid 116 may also allow the lamp light 106 to pass through the top of the cassette lid 116 to illuminate the microorganism colonies and/or to allow the fluorescence 120 to pass through the cassette lid 116 so that it remains perceptible by the photosensitive pixels 110. In some embodiments, the top surface of the cassette lid 116 may have a different optical property than the side surfaces of the cassette lid 116. The top surface may be configured to allow light from the lamps 104 and the fluorescence 120 to pass through, whereas the side surfaces may be configured to allow light from a side-mounted light source to pass through the cassette lid 116 and remain perceptible by a side-mounted sensor. The amount of transparency in the top surface may be selected based on the type of lamp 104 used, whereas the amount of transparency in the side surfaces may be selected based on the type or capabilities of side-mounted light source and/or side-mounted sensor.

In some embodiments, the cassette base 114 of the cassette 112 is substantially opaque. The cassette base may be considered substantially opaque when light from the side-mounted light source, which is capable of being perceived by the side-mounted sensor when it passes through the cassette lid 116, is not capable of being perceived by the side-mounted sensor when it passes through the cassette base 114. A substantially opaque cassette base 114 may also block background light from passing through the body of the cassette base 114.

### Automated Sample Processing System

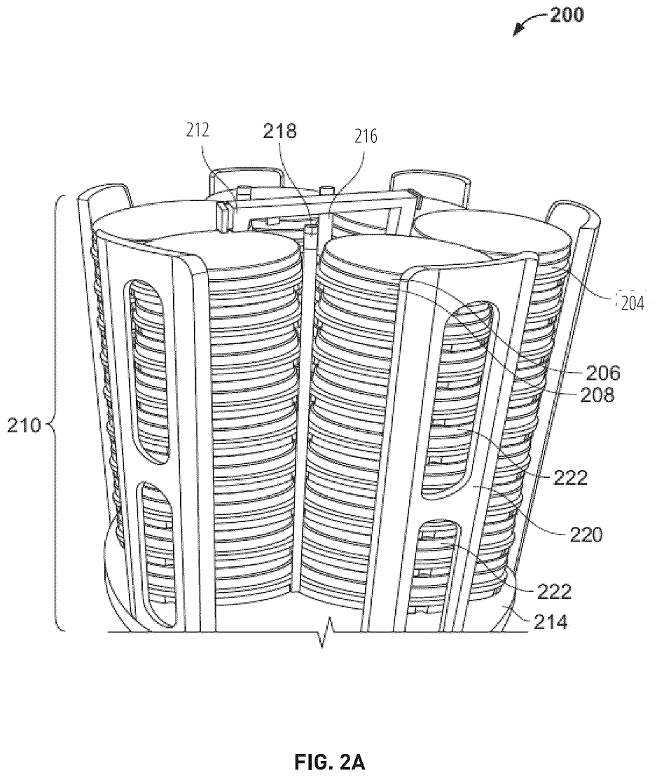

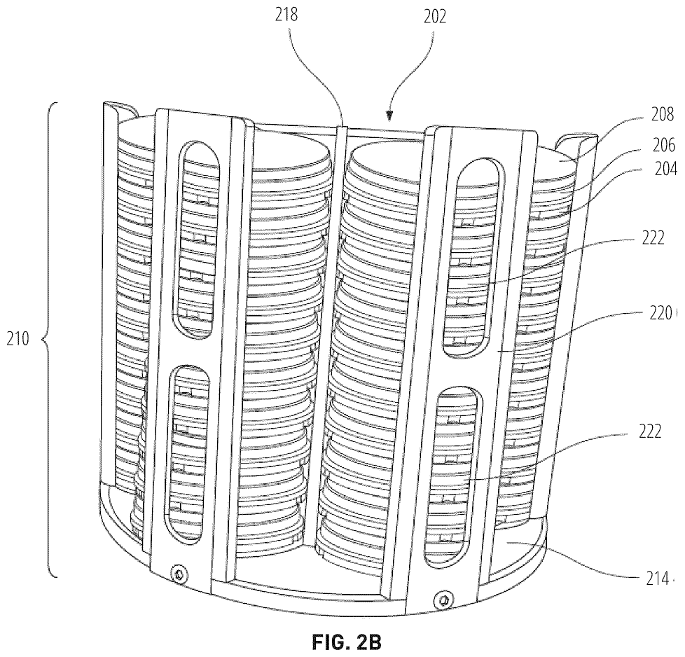

FIG. 2A and FIG. 2B illustrate examples of carousels 200, 202 suitable for use with a microbial detection and/or enumeration system. The carousel 200 may include cassettes 204 having cassette bases 206 and cassette lids 208, cassette columns 210, a carousel handle 212, a carousel base 214, a carousel stem 216, column posts 218, column supports 220, and column apertures 222.

An inferior end of the carousel stem 216 may be coupled to the superior face of the carousel base 214. The superior end of the carousel stem 216 may be coupled to the carousel handle 212. The cassette columns 210 may be coupled to the carousel base 214.

The carousel stem 216 may be any suitable shape; in the depicted example the carousel stem carousel stem 216 is substantially cylindrical. In other examples, the carousel stem 216 may be substantially rectangular or any shape capable of coupling to the carousel base 214 and the carousel handle 212.

In the depicted example, the carousel handle 212 is T-shaped. In other examples, the carousel handle 212 may be straight, D-shaped, a knob, a ring, or any shape capable to be gripped to lift the carousel 200. The lateral ends of the carousel handle 212 may extend longitudinally to couple to the carousel base 214, thereby forming an aperture between the longitudinally extended lateral end of the carousel handle 212 and the carousel stem 216.

In the depicted example, the carousel handle 212 is central to the carousel 200, surrounded by the at least one cassette column 210. In other examples, the carousel handle 212 need not be in the center of the incorrectly loaded carousel 200.

In the depicted example, the carousel base 214 is substantially circular. In other examples, the carousel base 214 may be substantially square, rectangular, triangular, pentagonal, hexagonal, heptagonal, or any other shape capable of holding at least one cassette column 210.

Each cassette column 210 may include zero or more column posts 218 and zero or more column supports 220. The column support 220 may include zero or more column apertures 222. The column apertures 222 may be evenly spaced or not evenly spaced. The column apertures 222 may be of any suitable shape, such as oval shaped, square, rectangular, circular, triangular, pentagonal, hexagonal, or any other shape.

The column support 220 may be substantially concave relative to the carousel stem 216. In other examples, the column support 220 may be a substantially straight surface or may be substantially convex relative to the carousel stem 216.

Each column post 218 may be positioned relatively internally and adjacent to the carousel stem 216 and the column support 220 may be positioned relatively externally with the outer edge of the column support 220 coupled to the superior perimeter of the carousel base 214. In other examples, each column post 218 may be positioned relatively externally with the outer edge of the column post 218 coupled to the superior perimeter of the carousel base 214 and the column support 220 may be positioned relatively internally and adjacent to the carousel stem 216.

Each cassette column 210 may share at least one column post 218 with an adjacent cassette column 210. Alternatively or in addition, some or all cassette columns 210 may share both column posts 218 with the adjacent cassette columns 210. In some examples, each cassette column 210 may include its own column posts 218. In some examples, the lateral ends of the carousel handle 212 may couple to column posts 218.

In the depicted embodiments, the carousel 200 includes six cassette columns 210, although the present invention is not limited to this configuration. Each cassette column 210 may have a capacity for zero or more cassettes 112 (the specific number of cassettes being dependent on the size of the carousel 200 and the size of the cassettes 112). The cassette columns cassette column 210 may be configured to each hold cassettes of the same size (e.g., having a radius of 55 mm, 90 mm, or any other suitable size. The cassette columns cassette column 210 may alternatively be configured so that different columns cassette column 210 may hold cassettes of different sizes.

In some embodiments, the carousels 200, 202 may be used in connection with a carousel elevator, which may be a device capable of raising and lowering the carousel base 214 longitudinally. In some embodiments, the carousel elevator raises the carousel base carousel base 214 longitudinally such that the cassette on top of the cassette column cassette column 210 is in a loaded position capable of being retrieved, manipulated, and/or analyzed by a microbial enumeration and/or detection system.

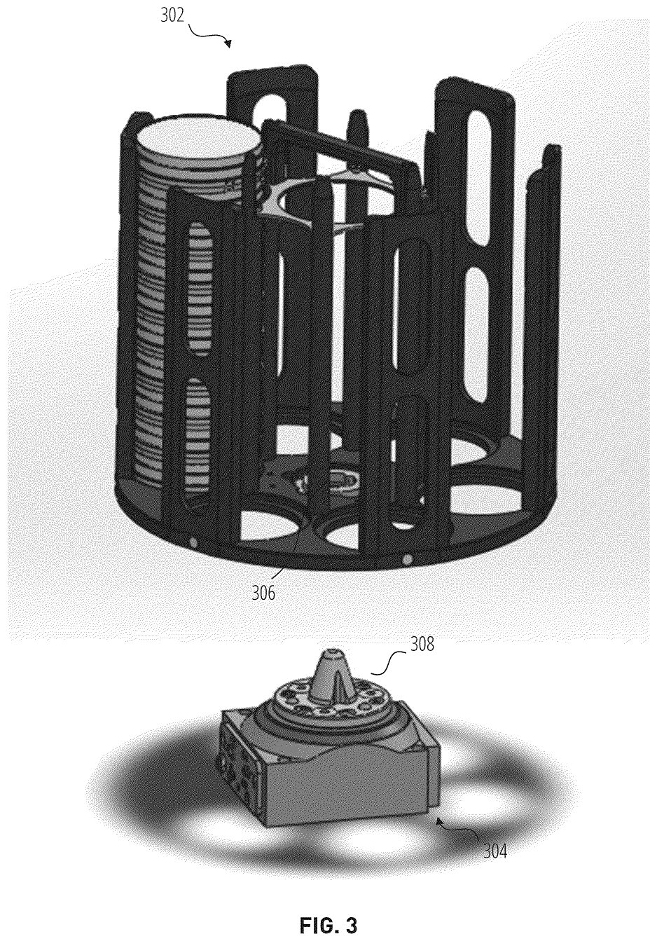

As the microbial enumeration and/or detection system needs to access different cassettes, it is possible that the cassettes will be located in different columns 210. Accordingly, carousel 200, 202 may be rotated so that the column cassette column 210 containing the next cassette for retrieval, manipulation, analysis, etc. is placed in a predetermined position. As previously noted, it is important that the carousel be rotatable into a precise and predictable location so the correct cassette can be retrieved. To that end, FIG. 3 depicts an example of a carousel 302 as it is being deployed on a rotatable indexer 304.

The indexer 304 may include a motor that rotates an indexer-side positioning element 308. The carousel may include a corresponding carousel-side positioning element 306 mounted to the carousel base, where the carousel-side positioning element 306 is capable of interfacing with the indexer-side positioning element 308. The carousel-side positioning element 306 and/or indexer-side positioning element 308 include features so that the carousel 302 can be initially correctly positioned on the indexer 304, and/or features to maintain the carousel 302 on the indexer 304 without slipping or otherwise moving relative to the indexer 304 during use. Accordingly, the carousel 302 can be rotated by the indexer 304 to precise, predetermined, and predictable locations to place a given column cassette column 210 where it is needed.

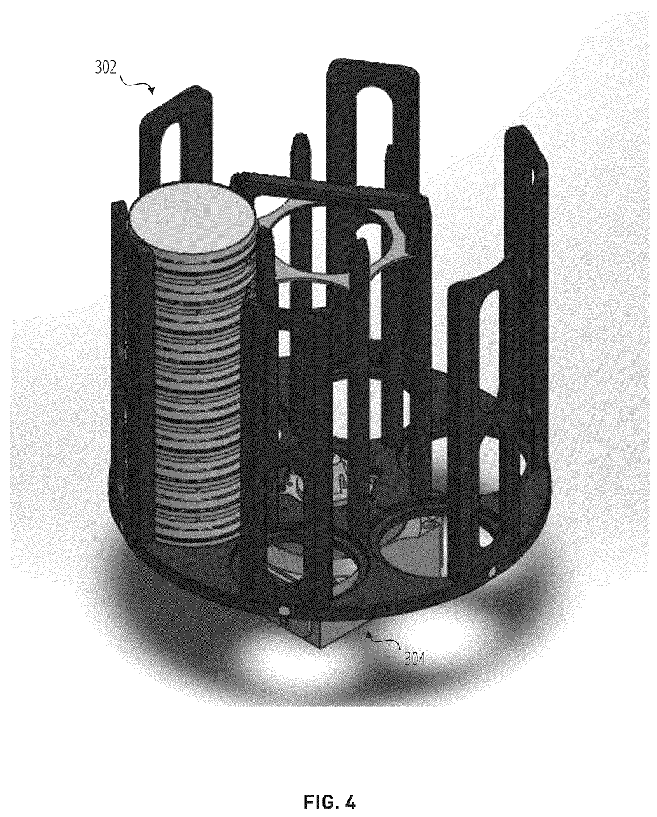

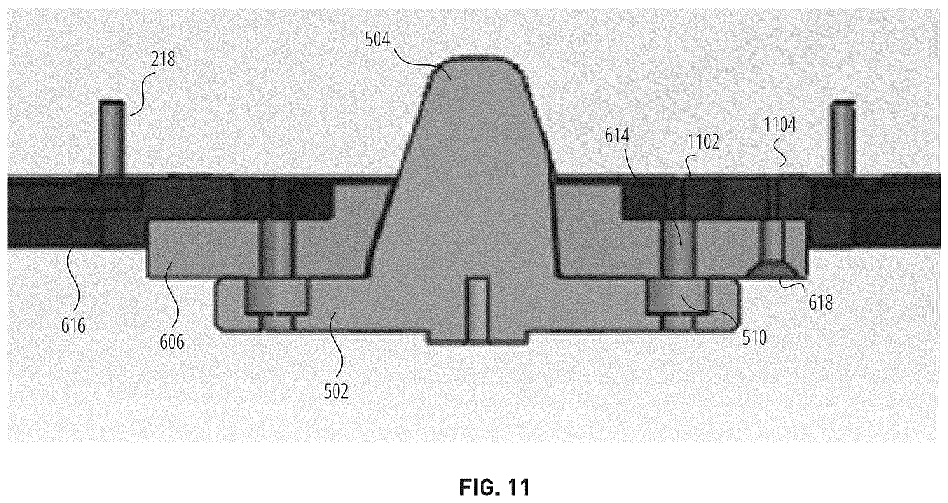

FIG. 4 shows the carousel 302 of FIG. 3 after it has been lowered onto the indexer 304. The carousel base may be fixed to the carousel-side positioning element 306, as can be seen more clearly in FIG. 11. The carousel-side positioning element 306 may be secured to the indexer-side positioning element 308 using any or a combination of ball bearings, magnets, mating features, and/or fixing elements such as bolts or screws, as will be discussed in more detail below. In some examples, fixing elements may be deployed that connect all of the indexer-side positioning element 308, the carousel-side positioning element 306, and the carousel base together as one unit.

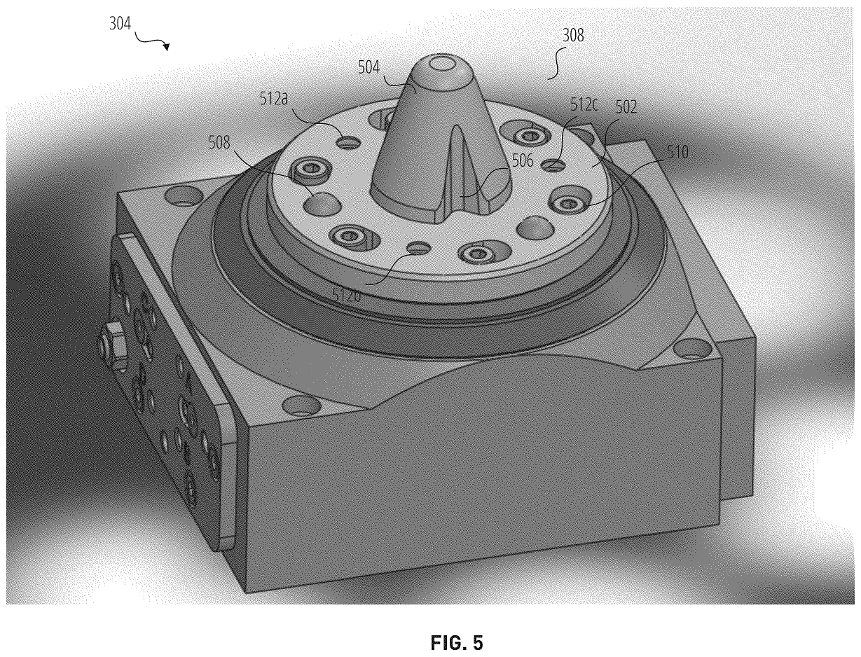

FIG. 5 shows the indexer 304, and particularly the indexer-side positioning element 308, in more detail.

The indexer-side positioning element 308 may include a spindle 504 or post that is formed integrally with, or attached to, an indexer-side plate 502. As will be described in more detail below, the spindle 504 may be sized and shaped to guide the carousel-side positioning element 306 into position on the indexer-side positioning element 308. To facilitate receiving and positioning the carousel-side positioning element 306, the spindle 504 may taper from a relatively narrow distal end spaced away from the indexer-side plate 502 to a relatively broad proximal end located proximal to the indexer-side plate 502. The spindle 504 may include one or more grooves 506 sized and configured to mate with corresponding protrusions and grooves in the carousel-side positioning element 306 so that the carousel 302 can be precisely positioned with little effort as it is lowered onto the indexer 304. Although the depicted example shows a single central spindle 504, the present invention is not so limited. An indexer-side positioning element 308 may include more than one spindle 504 arrayed in any suitable configuration for receiving the carousel-side positioning element 306.

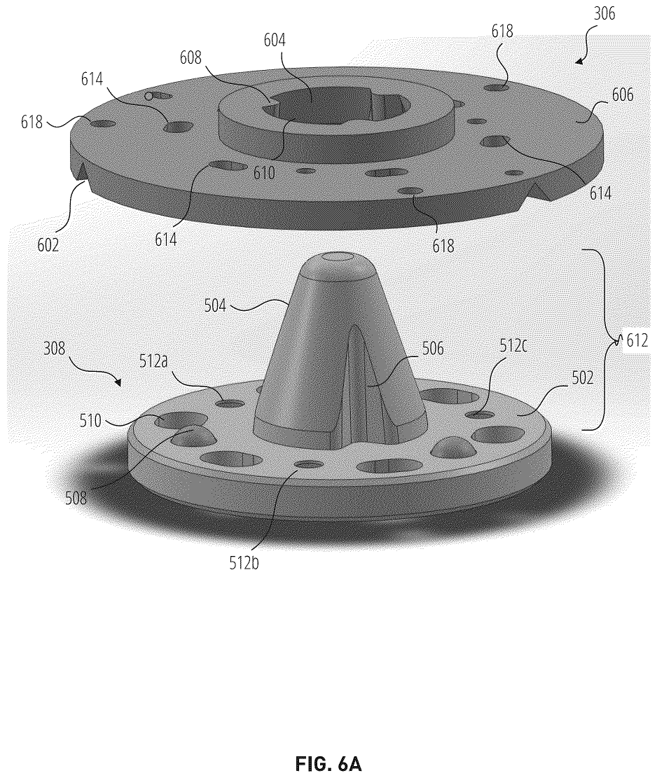

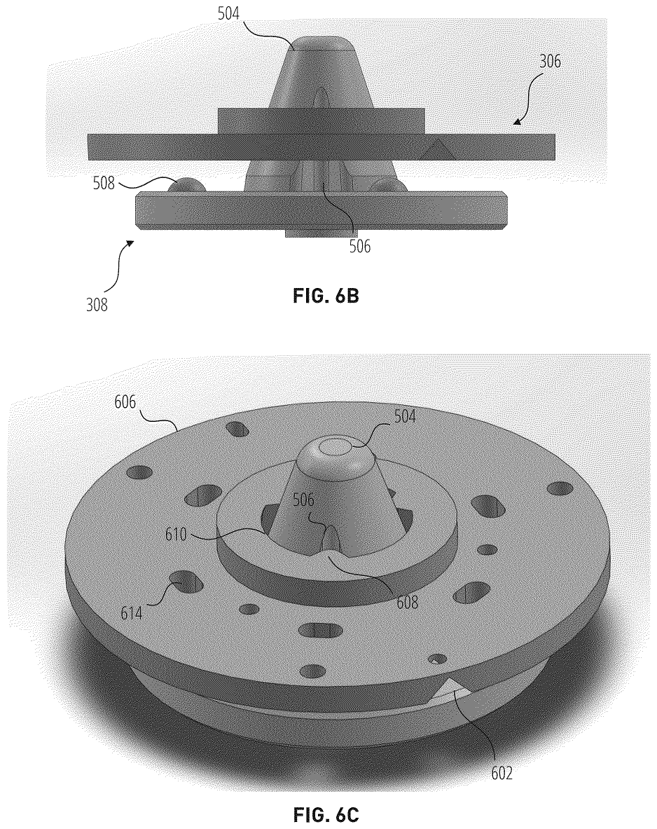

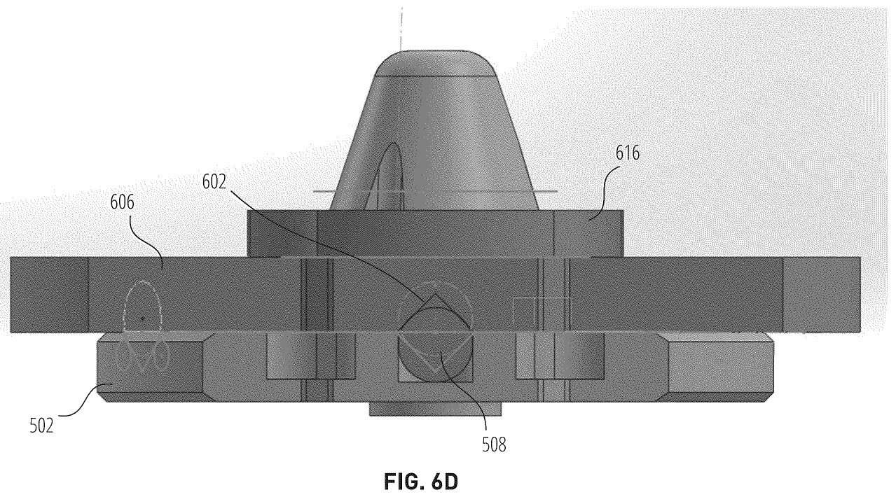

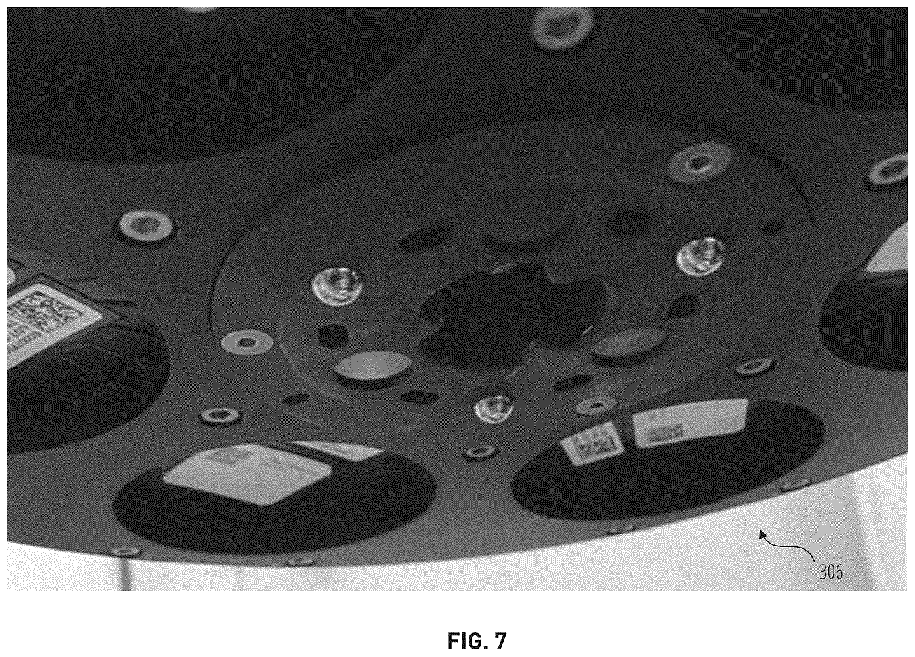

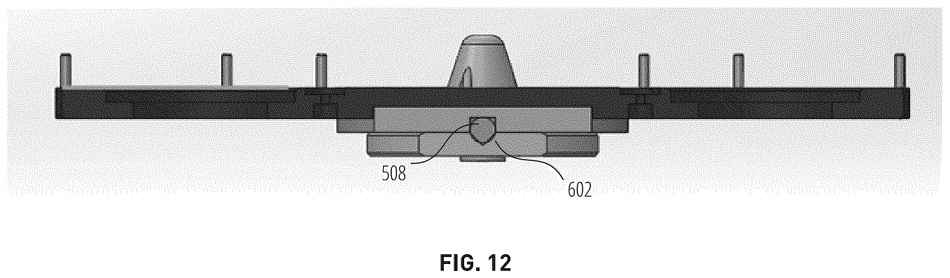

The indexer-side plate 502 of the indexer-side positioning element 308 may include one or more ball bearings 508. The ball bearing 508 may be sized and positioned on the indexer-side plate 502 to mate with corresponding grooves in the carousel-side positioning element 306, as will be shown in FIG. 6A-FIG. 6D and FIG. 12. When the carousel-side positioning element 306 is lowered onto the indexer-side positioning element 308, the grooves of the carousel-side positioning element 306 guide the ball bearings 508 into the correct position so that the carousel 302 can be placed precisely on the indexer 304. They also provide a degree of resistance that prevents accidental movement of the carousel 302 relative to the indexer 304. For example, if a force is applied tp the carousel 302 from the bottom, causing the carousel 302 to rise off the indexer 304, then the ball bearings 508 may be guided by the shape (e.g., triangular) of the grooves as the carousel 302 drops back onto the indexer 304 due to gravity, thus allowing the carousel 302 to be repositioned correctly on the indexer 304. In another example, if the carousel 302 is subjected to an external unintended force that would tend to cause the carousel 302 to rotate undesirably relative to the carousel 302, the presence of the ball bearings 508 in the grooves may allow the carousel 302 to resist the force and/or return to its original position due to the shape (e.g., triangular) of the grooves.

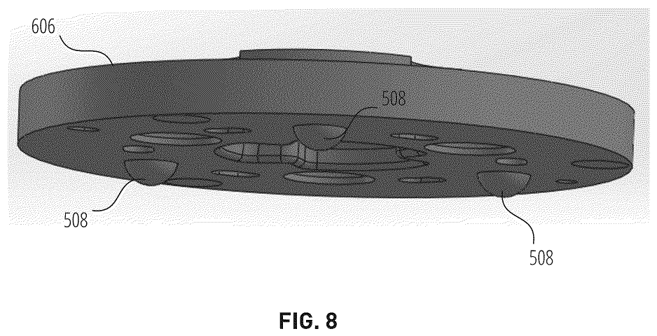

The example depicted in FIG. 5 through FIG. 6D shows ball bearings 508 on the indexer-side positioning element 308 and corresponding grooves in the carousel-side positioning element 306. However, one of ordinary skill in the art will recognize that the locations of the ball bearings 508 and grooves may be swapped so that the grooves are provided on the indexer-side positioning element 308 and the ball bearings 508 are provided on the carousel-side positioning element 306. An example of this configuration is shown in FIG. 7 through FIG. 10. The functionality of the ball bearings 508 and grooves remains the same in both examples. One advantage of placing the ball bearings 508 on the indexer-side positioning element 308, rather than on the carousel-side positioning element 306, is that it allows the carousel-side positioning element 306 to have a flat bottom. The carousel 302 is not always mounted to the indexer 304, and in many situations may be picked, moved around a laboratory space, and placed on different surfaces. By locating the ball bearings 508 on the indexer-side positioning element 308, the carousel base can be kept flat, without the ball bearings 508 extending therefrom. This may provide a more stable surface when the carousel 302 is placed on flat surfaces and not mounted to the indexer 304.

In a similar way, it is conceivable that the spindle 504 could be located on the carousel-side positioning element 306 rather than the indexer-side positioning element 308. For example, the spindle 504 could be inverted so that it tapers from a relatively wide configuration proximal to the carousel base to a relatively narrow configuration at a distal end. Such a spindle could be received in a corresponding opening of the indexer-side positioning element 308. However, this configuration would provide a greater obstacle on the base of the carousel (even as compared to the configuration in which the ball bearings 508 are placed on the carousel-side positioning element 306), which could make it difficult to place the carousel on flat surfaces.

However, these problems could be mitigated by providing multiple spindles arrayed around the carousel-side positioning element 306, especially if the spindles were made relatively short to reduce problems of balance. Alternatively or in addition, a specially-designed adapter base may be attached to the bottom of the carousel-side positioning element, where a top surface of the adapter would be configured to receive and interface with the bottom surface of the carousel-side positioning element 306 and the bottom surface of the adapter would be flat. When the carousel is placed on flat surfaces in the lab, it may be deployed with the adapter. The adapter may be removed when it is time to place the carousel on the indexer 304. Still further, the carousel-side positioning element 306 may be fixed to the carousel base in such a way that it is relatively easily removable (e.g., with clips, a snap-fit, a twist-lock, etc.) so that the carousel-side positioning element 306 can be removed from the carousel when the carousel is not deployed on the indexer 304.

The indexer-side plate 502 may include one or more optional fastener receivers 510, which may be through-holes sized and shaped to receive one or more fasteners (bolts, screws, etc.) to allow the indexer-side plate 502 to be fastened to the indexer 304 and/or to the carousel-side positioning element 306. The fastener receivers 510 are shown from the side in the cutaway view of FIG. 11.

Alternatively or in addition, the spindle 504 may include a suitable through-hole or post configured to mate with a corresponding feature on the indexer 304 (see FIG. 11). The corresponding features may mate so that the spindle 504 can be lowered onto the indexer and rotated as the feature of the indexer rotates.

The indexer-side plate 502 may also optionally include one or more magnets, such as a magnet 512a, a magnet 512b, and a magnet 512c. Although FIG. 5 depicts three magnets spaced equally around the indexer-side plate 502, one of ordinary skill in the art will recognize that this is only one possible configuration; more or fewer magnets may be employed in any suitable configuration. The magnets 512a, 512b, 512c may be configured to attract corresponding magnets on the carousel-side positioning element 306 to releasably lock the indexer-side positioning element 308 to the carousel-side positioning element 306.

Although the described example provides corresponding magnets on both the carousel-side positioning element 306 and indexer-side positioning element 308, this configuration is not required. For example, magnets may be deployed on only one of the carousel-side positioning element 306 or indexer-side positioning element 308, with the other element being either made of a ferrous material or having a ferrous material attached or embedded at suitable corresponding locations so that the magnets attract the ferrous metal.

An attractive strength of the magnets may be selected so that the carousel-side positioning element 306 remains locked to the indexer-side positioning element 308 in the presence of the typical force of rotation applied by the indexer 304 and/or undesirable (e.g., accidental) forces that would tend to knock the carousel 302 out of alignment on the indexer 304. The attractive force may also be selected not to be so great so that an average user is not capable of separating the carousel-side positioning element 306 from the indexer-side positioning element 308 when the carousel 302 is intentionally lifted from the indexer 304. Alternatively or in addition, electromagnets may be used in place of the magnets. This may allow the magnetic force to be turned on and off as needed, and further may allow the force to be varied (e.g., depending on the rotation speed of the indexer, the weight of the carousel, the strength of the user attempting to separate the carousel-side positioning element from the indexer-side positioning element, etc.

Although the depicted embodiment utilizes fasteners, ball bearings, and magnets in combination to fix the carousel-side positioning element 306 to the indexer-side positioning element 308, each of these securing mechanisms may be deployed individually or in any combination. Other types of fasteners, such as adhesives, a high-friction surface, physical mating features on the carousel-side positioning element 306 and indexer-side positioning element 308 etc. may also or alternately be used.

FIG. 6A-FIG. 6B depict the indexer-side positioning element 308 in combination with a corresponding carousel-side positioning element 306 as the carousel-side positioning element 306 is lowered onto the indexer-side positioning element 308. For purposes of illustration, the carousel-side positioning element 306 is shown isolated. In practice, the carousel-side positioning element 306 would typically be attached to the bottom of the carousel 302 during this process.

The carousel-side positioning element 306 includes a carousel-side plate 606 configured to be attached to a carousel base 616 of the carousel 302.

The carousel-side plate 606 includes grooves 602 configured to receive the ball bearings 508 of the indexer-side positioning element 308. In the depicted example, the grooves 602 have a triangular cross section to interface with the spherical ball bearings 508 and guide the carousel-side positioning element 306 and indexer-side positioning element 308 into position relative to each other. One of ordinary skill in the art will recognize that other shapes (round, rectangular, etc.) may also be used.

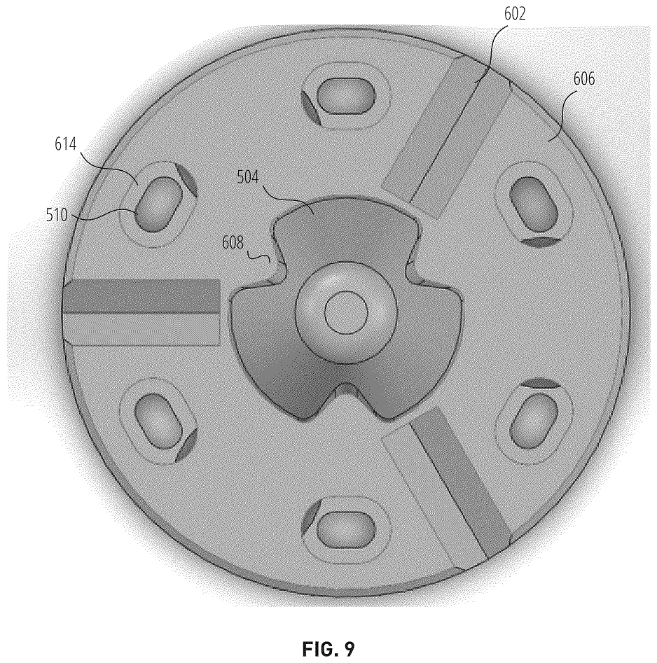

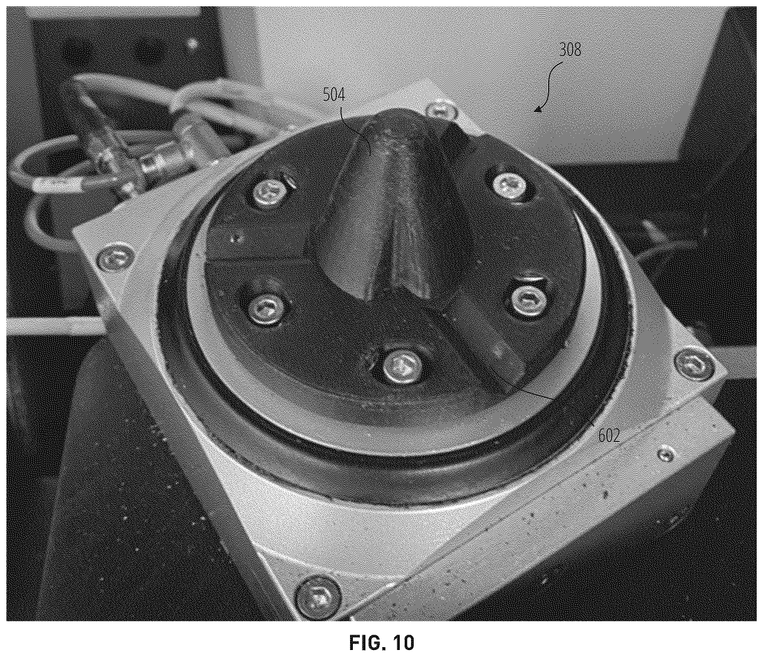

The carousel-side plate 606 further includes an opening 604 sized, shaped, and positioned to receive the spindle 504 of the indexer-side positioning element 308. The opening 604 may include an alternating series of protrusions 608 and recesses 610, with the protrusion 608 configured to interface with the grooves 506 of the spindle 504 (and the groove-less areas of the spindle 504 fitting into the recesses 610). For example, the protrusions 608 and grooves 506 may be sized so that a radially inner-most point of the protrusion 608 is positioned within a radially-outermost extent of the groove 506 when the carousel-side plate 606 is initially lowered onto the indexer-side plate 502. In other words, the carousel-side plate 606 will not fully drop onto the indexer-side plate 502 if the protrusions 608 are not aligned to the grooves 506, because the protrusion 608 will instead contact and be blocked by the groove-less portion of the spindle 504. When properly positioned, the protrusion 608 may be just inside the groove 506. As the carousel-side positioning element 306 slides down the spindle 504, the protrusions 608 extend further into the grooves 506, until (when the carousel-side positioning element 306 is in face-to-face contact with the indexer-side positioning element 308), the protrusions 608 are flush with (or very close to) the walls of the grooves 506 (see FIG. 9). In this way, the carousel-side positioning element 306 can be initially properly aligned to the indexer-side positioning element 308 as the carousel 202 is lowered onto the indexer 304, and the grooves 506 and protrusions 608 serve to guide the carousel-side positioning element 306 into position as it is lowered.

Although the depicted examples show an opening 604 in the carousel-side plate 606 with protrusion 608 configured to mate with grooves 506 on the spindle 504, the opposite configuration may also be employed. In such an example, the carousel-side plate 606 may include an opening with grooves configured to mate with corresponding protrusions on the spindle 504.

The spindle extends away from the indexer-side plate 502 by a distance representing a spindle height 612. The spindle height 612 may be selected to be large enough so that the carousel-side plate 606, when placed over the spindle 504, can be effectively guided down onto the indexer-side plate 502 without the need for the user to visually verify the positioning of the carousel-side plate 606 with respect to the indexer-side plate 502. However, the spindle height 612 may also be selected, depending on the application and location of use of the indexer 304, so that the spindle 504 does not extend so high that the user would need to lift the carousel 302 to an uncomfortable height in order to load the carousel 302 on the spindle 504. For example, accounting for the average user's arm length and reach, as well as the expected height of the indexer 304 above the floor, the spindle height 612 may be selected so that the user can comfortably lift the carousel 302 by its handle to a height sufficient for the carousel base 616 to clear the spindle 504 and be easily lowered onto the spindle 504.

The carousel-side plate 606 may also include through-holes sized and positioned to receive fastening elements, such as screws, bolts, pins, etc. The through-holes may be located at different positions on the carousel-side plate 606, depending on the elements that are being fastened together. For example, the 606 includes fastener through-holes 614 located on a (relatively) radially interior part of the carousel-side plate 606, through which fasteners can be passed to secure the carousel-side plate 606 to the indexer-side plate 502 and/or a radially interior portion of the carousel 202 (see FIG. 11). Alternatively or in addition, through-holes 618 may be located on a (relatively) radially exterior part of the carousel-side plate 606. In this position, the through-holes 618 may be in a position so that they are not covered by the indexer-side plate 502 (i.e., the maximum radius of the indexer-side plate 502 is less than the distance from the center of the carousel-side plate 606 to the through-holes 618 so that, when the carousel-side plate 606 is placed on top of the indexer-side plate 502, the through-holes 618 remain unobstructed by the indexer-side plate 502 when the carousel-side plate 606 is accessed from the bottom; see FIG. 11 for an example). One or more fasteners may be passed through the through-holes 618 to secure the carousel-side plate 606 to the carousel 202.

In some embodiments, the through-holes on the indexer-side plate 502, the carousel-side plate 606, and/or the carousel base 214 may be positioned so that, when the carousel-side positioning element 306 and the indexer-side positioning element 308 are properly aligned (e.g., through the grooves, ball bearings, protrusions, and/or magnets), the through-holes line up with each other. In some examples, as shown for instance in FIG. 11, the corresponding through-holes on the various elements may be of different sizes (e.g., of different diameters). This may allow a user to use, for example, a fastener with a hollow center in which another fastener may be received. For instance, the fastener receiver 510 may be sized and shaped to receive a relatively large fastener, which is passed down through the indexer-side plate 502 from the top to secure the indexer-side plate 502 to the indexer 304. The fastener may include a relatively large top with a threaded or non-threaded opening (see, e.g., FIG. 5 and FIG. 10) into which another fastener may be passed.

As shown in FIG. 9 and FIG. 11, the fastener receiver 510 is positioned (when the plates are properly aligned) in a location corresponding to the fastener through-hole 614 of the carousel-side plate 606. The diameter of the fastener through-hole 614 is less than the diameter of the fastener receiver 510, and is sized to correspond to the opening in the fastener that is passed through the fastener receiver 510. A second fastener may be passed through the fastener through-hole 614 and into the opening of the fastener in the fastener receiver 510 to secure the carousel-side plate 606 to the indexer-side plate 502. For example, the second fastener may be a pin, screw, bolt, etc. In some examples, the second fastener may be a protrusion or extension of the cassette carousel base 616, which passes through the bottom of the carousel-side plate 606 and extends into the opening in the fastener in the fastener receiver 510 when the carousel-side positioning element 306 and indexer-side positioning element 308 are aligned and the carousel 202 is lowered onto the indexer 304. In others, the carousel base 616 may include carousel through-hole 1102, 1104 to allow fasteners to be passed through the carousel base 616 and into the fasteners in the carousel-side plate 606 in a manner similar to the one discussed above for mating the carousel-side plate 606 to the indexer-side plate 502.

FIG. 7 and FIG. 8 depict another example of a carousel-side positioning element 306. The carousel-side positioning element 306 in FIG. 7 and FIG. 8 is similar to the carousel-side positioning element 306 of the preceding Figures, except that the ball bearings 508 are located on the carousel-side plate 606 instead of the indexer-side plate 502. As discussed above, corresponding grooves for receiving the ball bearings 508 may be provided in the indexer-side plate 502. FIG. 12 provides a cutaway side-view of this configuration.

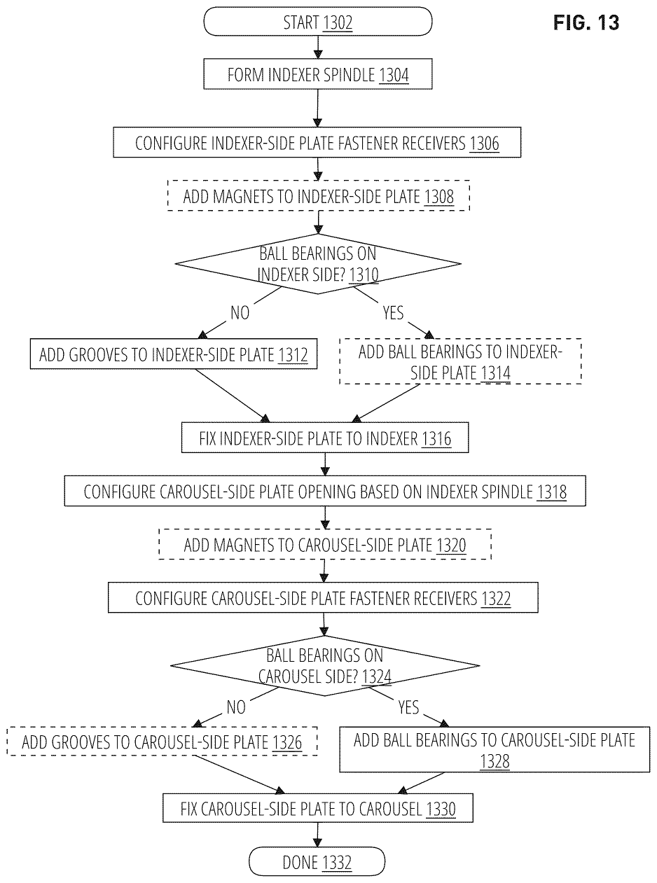

FIG. 13 illustrates an example routine for manufacturing or assembling an alignment system for a microbial detection and/or enumeration system, the alignment system including an indexer-side positioning element and a carousel-side positioning element. Although the example routine depicts a particular sequence of operations, the sequence may be altered without departing from the scope of the present disclosure. For example, some of the operations depicted may be performed in parallel or in a different sequence that does not materially affect the function of the routine. In other examples, different components of an example device or system that implements the routine may perform functions at substantially the same time or in a specific sequence.

According to some examples, the method includes starting at start block 1302.

According to some examples, the method includes forming indexer spindle at block 1304. The indexer spindle 504 may be a separate piece configured to be attached to an indexer-side plate 502, or may be formed integrally with the indexer-side plate 502. If separate, then the spindle 504 and indexer-side plate 502 may be separately formed in block 1304 and the indexer-side plate 502 may be affixed to the indexer-side plate 502 with fasteners, adhesive, welding, or any other suitable attachments; if integral, then the spindle 504 may be formed as part of the same process of forming the indexer-side plate 502 at block 1304. The spindle 504 and/or indexer-side plate 502 may be formed by additive and/or subtractive manufacturing techniques; for example, the spindle 504 and/or indexer-side plate 502 may be machined or tooled to remove material (e.g., from the areas representing the grooves 506). Alternatively or in addition, the spindle 504 and/or indexer-side plate 502 may be formed by a 3D printing (or similar) process.

According to some examples, the method includes configuring indexer-side plate fastener receivers at block 1306. The indexer-side plate 502 fastener receivers 510 may be in the form of through-holes which may be drilled into the indexer-side plate 502 at this step. Alternatively or in addition, the fastener receivers 510 may be formed into the indexer-side plate 502 as part of an additive manufacturing process. The fastener receivers 510 may be positioned at radial positions on the indexer-side plate 502 so as to mate with corresponding threaded holes, posts, or other features at corresponding radial positions on the indexer 304 so that the indexer-side plate 502 can be secured to the indexer 304. As an alternative, the indexer-side plate 502 and/or spindle 504 may be formed integrally with the indexer 304, such that fasteners, adhesives, etc. are not required to mount the indexer-side positioning element 308 to the indexer 304.

According to some examples, the method optionally includes adding magnets to indexer-side plate at block 1308. If magnets are to be used, they may be located on the indexer-side plate 502, the carousel-side plate 606, or both. If located on the indexer-side plate 502, they may be positioned at appropriate locations distributed around the indexer-side plate 502 to both correspond to magnets or ferrous locations on the carousel-side plate 606 and to allow for an evenly-distributed securing force. If magnets are not desired on the indexer-side plate 502 but are desired on the carousel-side plate 606, then one or more ferrous plates may be attached to the indexer-side plate 502, embedded in the plate during manufacturing, or the plate may be formed mostly or entirely of a ferrous material.

According to some examples, the method includes evaluating whether ball bearings should be applied on indexer side at decision block 1310. In some examples, ball bearings are not provided (in which case the answer at decision block 1310 is “NO”); in others, the ball bearings may be provided on the carousel side (in which case the answer at decision block 1310 is also “NO”).

If ball bearings are to be added to the indexer side (“YES” at decision block 1310) then suitable cavities for the ball bearings may be formed in the indexer-side plate 502 during the fabrication process (block 1304, for example) and ball bearings may be added to the cavities at block 1314 and may be secured (e.g., by forming a lip on the cavities to hold the ball bearings in place. Alternatively, the ball bearings may be mounted in a cage that is secured in the cavities, among other mounting options that will be known to those of ordinary skill in the art.

If ball bearings are not to be added to the indexer side at decision block 1310 (and ball bearings are instead to be added on the carousel-side plate 606), then grooves for the ball bearings may instead be added to the indexer-side plate 502 at block 1312. The grooves may be formed during an additive manufacturing processed, may be removed from the indexer-side plate 502 by subtractive manufacturing, or any other suitable method. In one example, the grooves have a triangular cross-section for accommodating the ball bearings 508 and guiding the carousel-side positioning element 306 and indexer-side positioning element 308 into position, although this shape is not required. Other groove shapes may include circular, parabolic, square, rectangular, trapezoidal, etc. cross-sections. The grooves may be arrayed around the indexer-side plate 502 and may extend radially from the spindle 504 to the outer circumference of the indexer-side plate 502; alternatively, the grooves may extend only partially along the radius of the indexer-side plate 502.

According to some examples, the method includes fixing indexer-side plate 502 to indexer 304 at block 1316. For example, the indexer-side plate 502 may be positioned on the indexer 304 (e.g., by passing a post on the indexer 304 into the opening in the spindle 504, discussed above) and fasteners may be passed through the fastener receivers formed in block 1306. The fasteners may be screwed into threaded holes in the indexer 304 located at positions corresponding to the through-holes. In some examples, the fasteners may include openings for receiving a further fastener passed through the top of the indexer-side plate 502. In some examples, the indexer-side plate is formed integrally with the indexer, in which case it may not be necessary to mount the indexer-side plate 502 to the indexer 304.

Next, a technique for forming the carousel-side plate 606 is described. The carousel-side plate 606 may be formed by additive and/or subtractive manufacturing processes, and may be formed using the same or different techniques as compared to the indexer-side plate 502. The carousel-side plate 606 may be formed to be substantially round and with a sufficient thickness (depending on the material used) to support the carousel 202 and to avoid deformation when the carousel 202 rests on the carousel-side plate 606 and when the carousel 202 is rotated on the indexer 304.

According to some examples, the method includes configuring a carousel-side plate opening 604 based on indexer spindle at block 1318. The opening 604 may be formed with protrusions 608 corresponding to the locations of the grooves 506 in the spindle 504, and may be sized as described above.

The remainder of the process for forming the carousel-side plate 606 generally corresponds to the similar blocks discussed above for forming the indexer-side plate 502. At block 1320, magnets and/or ferrous materials may be added to the carousel-side plate 606 at appropriate locations. At block 1322, fastener receivers may be added to the carousel-side plate 606. At block 1324, it may be determined if the ball bearings are to be located on the carousel side. If so, then at block 1328 ball bearings may be added to the carousel-side plate 606; if not, then at block 1326 grooves may optionally be added to the carousel-side plate 606 for receiving ball bearings located on the indexer-side plate 502. At block 1330, the carousel-side plate 606 may be fixed to the carousel 302 using suitable fasteners passed through the fastener receivers. In light of the corresponding description already given with respect to block 1304 to block 1316, further discussion is omitted here for the sake of brevity.

According to some examples, the method terminates at done block 1332.

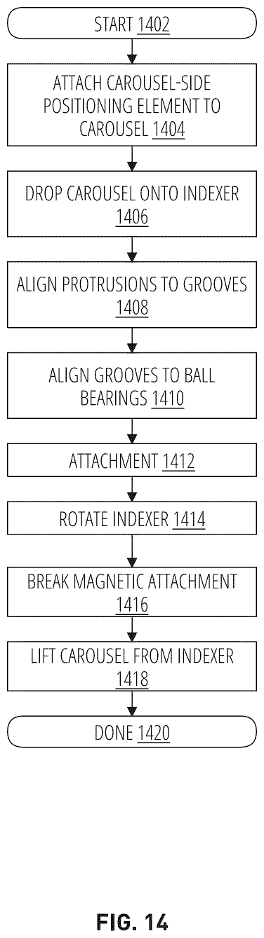

FIG. 14 illustrates an example routine for deploying an alignment system in a microbial detection and/or enumeration system, the alignment system including an indexer-side positioning element and a carousel-side positioning element. Although the example routine depicts a particular sequence of operations, the sequence may be altered without departing from the scope of the present disclosure. For example, some of the operations depicted may be performed in parallel or in a different sequence that does not materially affect the function of the routine. In other examples, different components of an example device or system that implements the routine may perform functions at substantially the same time or in a specific sequence.

According to some examples, the method includes starting at start block 1402.

According to some examples, the method includes attaching a carousel-side positioning element 306 to a carousel 302 at block 1404. The carousel-side positioning element 306 may include a carousel-side plate 606 with one or more through-holes 618 through which fasteners can be passed through the carousel-side plate 606 and into the carousel base 616 (or vice versa). At block 1404, the fasteners may be used to secure the carousel-side positioning element 306 to the carousel 302.

According to some examples, the method includes dropping or lowering the carousel 302 onto the indexer 304 at block 1406. A user may initially manually align the protrusion 608 in the opening 604 of the carousel-side plate 606 with the grooves 506 on the spindle 504 of the indexer 304, at block 1408 the carousel at block 1408. As the carousel 302 drops onto the indexer 304 through the action of the user and/or gravity, the protrusions 608 and grooves 506 may automatically maintain the carousel 302 and indexer 304 in alignment with each other.

As the carousel-side plate 606 approaches the indexer-side plate 502, ball bearings in either plate may pass into grooves in the other plate. Due to the shape of the grooves (e.g., triangular), the two plates may be further moved into (and held in) appropriate alignment.

If magnets are used on one or both plates, at block 1412 the magnets and/or ferrous materials may engage with each other as the carousel-side plate 606 approaches the indexer-side plate 502. The attraction of the magnets/ferrous materials may serve to releasably lock the indexer-side plate 502 to the carousel-side plate 606. Alternatively or in addition, clips or other securing mechanisms may be used to fasten the two plates together at block 1412.

The indexer 304 may then be rotated at block 1414. The rotation of the indexer may be controlled by a processor or controller, which receives a command signal based on a cassette that has been requested from the carousel. The processor/controller may identify a cassette column 210 on the carousel that holds the requested cassette, and may rotate the carousel 302 so that the cassette column 210 is in alignment with an appropriate tool of the microbial detection and/or enumeration system. For example, if the image is to be moved by a robotic arm, the carousel 302 may be rotated so that the cassette is at a location where the robotic arm can reach. If the cassette is to be imaged, the carousel 302 may be rotated to place the cassette column 210 holding the cassette 204 under a camera or other sensor.

When the carousel 302 is to be removed from the indexer 304, then at block 1416 the magnetic attachment may be broken. This may be achieved for example, by exerting an upward force on the carousel 302 sufficient to overcome the force of attraction of the magnets. For example, a user may pick up the carousel 302 by the carousel handle 212, pulling upwards away from the indexer 304 with sufficient force to break the carousel 302 away from the indexer-side plate 502. The user may then lift the carousel 302 from the indexer 304 at block 1418, allowing the carousel 302 to be moved to a new location and/or for a different carousel 302 to be loaded onto the indexer 304.

According to some examples, the method terminates at done block 1420.

It will be appreciated that the exemplary devices shown in the block diagrams described above may represent one functionally descriptive example of many potential implementations. Accordingly, division, omission or inclusion of block functions depicted in the accompanying figures does not infer that the hardware components, circuits, software and/or elements for implementing these functions would necessarily be divided, omitted, or included in embodiments.

Some embodiments may be described using the expression “one embodiment” or “an embodiment” along with their derivatives. These terms mean that a particular feature, structure, or characteristic described in connection with the embodiment is included in at least one embodiment. The appearances of the phrase “in one embodiment” in various places in the specification are not necessarily all referring to the same embodiment. Moreover, unless otherwise noted the features described above are recognized to be usable together in any combination. Thus, any features discussed separately may be employed in combination with each other unless it is noted that the features are incompatible with each other.

Some embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment. In the appended claims, the terms “including” and “in which” are used as the plain-English equivalents of the respective terms “comprising” and “wherein,” respectively. Moreover, the terms “first,” “second,” “third,” and so forth, are used merely as labels, and are not intended to impose numerical requirements on their objects.

What has been described above includes examples of the disclosed architecture. It is, of course, not possible to describe every conceivable combination of components and/or methodologies, but one of ordinary skill in the art may recognize that many further combinations and permutations are possible. Accordingly, the novel architecture is intended to embrace all such alterations, modifications and variations that fall within the spirit and scope of the appended claims.

Examples of the alignment system and techniques for its manufacture and use may be encompassed by the following:

1. A system for use with a microbial detection and/or enumeration system comprising a carousel configured to hold one or more cassettes and to be rotated into predefined positions by an indexer, the system comprising:
a carousel-side positioning element configured to be attached to the carousel; and
an indexer-side positioning element configured to be attached to the indexer, wherein
the carousel-side positioning element is configured to interface with the indexer-side positioning element to guide the carousel into a predetermined position when the carousel is deployed on the carousel.

2. The apparatus of claim 1, wherein the indexer-side positioning element comprises an indexer-side plate and a spindle, the spindle sized and shaped to be received in a corresponding opening of the carousel-side positioning element.

3. The apparatus of claim 2, wherein the spindle comprises one or more grooves and the opening of the carousel-side positioning element comprises one or more protrusions extending into the opening, wherein the protrusions are sized, shaped, and positioned in the opening to interface with the grooves of the spindle as the carousel-side positioning element is lowered onto the indexer-side positioning element.

4. The apparatus of claim 2 or 3, wherein the spindle tapers from a distal end to a proximal end to guide the carousel-side positioning element into place as the carousel-side positioning element is lowered onto the spindle.

5. The apparatus of claim 2 or 3, wherein the indexer-side plate comprises one or more ball bearings sized positioned to be received in corresponding grooves in the carousel-side positioning element to position the indexer-side positioning element with respect to the carousel-side positioning element.

6. The apparatus of any of the preceding claims, wherein the indexer-side positioning element includes one or more magnets or electromagnets and the carousel-side positioning element includes one or more corresponding magnets or electromagnets configured to attract the one or more magnets or electromagnets of the indexer-side positioning element.

7. The apparatus of any of the preceding claims, wherein one of the indexer-side positioning element or the carousel-side positioning element comprises one or more magnets or electromagnets, and the other of the indexer-side positioning element and the carousel-side positioning element includes one or more ferrous surfaces configured to attract the one or more magnets or electromagnets.

8. The apparatus of any of the preceding claims, wherein the carousel-side positioning element comprises a carousel-side plate configured to be attached to a base of the carousel, the carousel-side plate comprising an opening configured to receive a spindle of the indexer-side positioning element.

9. The apparatus of claim 8, wherein the carousel-side plate comprises one or more ball bearings sized positioned to be received in corresponding grooves in the indexer-side positioning element to position the indexer-side positioning element with respect to the carousel-side positioning element

10. The apparatus of any of the preceding claims, further comprising one or more fasteners configured to secure:
the indexer-side positioning element to the indexer;
the carousel-side positioning element to the carousel; or
the indexer-side positioning element to the carousel through the carousel-side positioning element.

11. An indexer for use with a microbial detection and/or enumeration system comprising a carousel configured to hold one or more cassettes and including an carousel-side positioning element configured to assist the indexer in rotating the carousel into predefined positions, the indexer comprising:
an indexer body housing a motor;
an indexer-side plate configured to be rotated by the motor; and
a spindle configured to receive the carousel-side positioning element, the spindle comprising one or more grooves configured to position the carousel with respect to the indexer as the carousel-side plate is lowered onto the spindle.

12. A carousel for use with a microbial detection and/or enumeration system comprising an indexer configured to rotate the carousel into predefined positions, the carousel comprising: one or more cassette columns;
a carousel base supporting the one or more cassette columns; and
a carousel-side positioning element configured to position the carousel with respect to the indexer, the carousel-side positioning element comprising an opening configured to receive a spindle of the indexer, the opening including one or more protrusions corresponding to grooves in the spindle.

13. A method of manufacturing a system for positioning a carousel with respect to an indexer configured to rotate the carousel into predefined positions, the method comprising:
forming an indexer spindle on an indexer-side plate, the indexer spindle extending from a proximal end closest to the indexer-side plate to a distal end and tapering from a relatively large diameter at the proximal end to a relatively small diameter at the distal end;
forming one or more grooves in the indexer spindle;
accessing a carousel-side plate; and
forming an opening in the carousel-side plate, the forming comprising shaping one or more protrusions in the opening to correspond to the one or more grooves of the indexer spindle.

14. The method of claim 13, further comprising adding magnets or electromagnets to one or more of the indexer-side plate or the carousel-side plate.

15. The method of any of claims 13-14, further comprising securing one or more ball bearings in the indexer-side plate and forming one or more grooves in the carousel-side plate configured to receive the ball bearings to guide the carousel-side plate onto the indexer-side plate.

16. The method of any of claims 13-15, further comprising securing one or more ball bearings in the carousel-side plate and forming one or more grooves in the indexer-side plate configured to receive the ball bearings and to guide the carousel-side plate onto the indexer-side plate.

17. The method of any of claims 13-16, further comprising: fixing the carousel-side plate to the carousel using one or more carousel-side fasteners; and fixing the indexer-side plate to the indexer using one or more indexer-side fasteners.

18. A method of using a system for positioning a carousel with respect to an indexer configured to rotate the carousel into predefined positions, the method comprising:
attaching a carousel-side positioning element to the carousel, the carousel-side positioning element comprising an opening;
attaching an indexer-side positioning element to the indexer, the indexer-side positioning element comprising a spindle configured to be received in the opening of the carousel-side positioning element; and
dropping the carousel-side positioning element onto the indexer-side positioning element, wherein dropping the carousel-side positioning element causes one or more protrusions in the opening of the carousel-side positioning element to be received in one or more corresponding grooves on the spindle to automatically align the carousel with the indexer.

19. The method of claim 18, further comprising aligning one or more magnets or electromagnets on the carousel-side positioning element to one or more corresponding magnets or electromagnets on the indexer-side positioning element to secure the carousel to the indexer.

20. The method of any of claims 18-19, further comprising aligning one or more ball bearings on one of the carousel-side positioning element or the indexer-side positioning element to one or more grooves of the other of the carousel-side positioning element or the indexer-side positioning element.

### LISTING OF DRAWING ELEMENTS

202 carousel
204 cassette
206 base
208 cassette lid
210 cassette column
212 carousel handle
214 carousel base
216 carousel stem
218 column post
220 column support
222 column aperture
302 carousel
304 indexer
306 carousel-side positioning element
308 indexer-side positioning element
502 indexer-side plate
504 spindle
506 groove
508 ball bearing
510 fastener receiver
512a magnet
512b magnet
512c magnet
602 groove
604 opening
606 carousel-side plate
608 protrusion
610 recess
612 spindle height
614 fastener through-hole
616 carousel base
618 through-hole
1102 carousel through-hole
1104 carousel through-hole

What is claimed is:

1. A system for use with a microbial detection and/or enumeration system comprising a carousel configured to hold one or more cassettes and to be rotated into predefined positions by an indexer, the system comprising:
a carousel-side positioning element configured to be attached to the carousel; and
an indexer-side positioning element configured to be attached to the indexer, wherein
the carousel-side positioning element is configured to interface with the indexer-side positioning element to guide the carousel into a predetermined position when the carousel is deployed on the carousel.

2. The apparatus of claim 1, wherein the indexer-side positioning element comprises an indexer-side plate and a spindle, the spindle sized and shaped to be received in a corresponding opening of the carousel-side positioning element.

3. The apparatus of claim 2, wherein the spindle comprises one or more grooves and the opening of the carousel-side positioning element comprises one or more protrusions extending into the opening, wherein the protrusions are sized, shaped, and positioned in the opening to interface with the grooves of the spindle as the carousel-side positioning element is lowered onto the indexer-side positioning element.

4. The apparatus of claim 2, wherein the spindle tapers from a distal end to a proximal end to guide the carousel-side positioning element into place as the carousel-side positioning element is lowered onto the spindle.

5. The apparatus of claim 2, wherein the indexer-side plate comprises one or more ball bearings sized positioned to be received in corresponding grooves in the carousel-side positioning element to position the indexer-side positioning element with respect to the carousel-side positioning element.

6. The apparatus of claim 1, wherein the indexer-side positioning element includes one or more magnets or electromagnets and the carousel-side positioning element includes one or more corresponding magnets or electromagnets configured to attract the one or more magnets or electromagnets of the indexer-side positioning element.

7. The apparatus of claim 1, wherein one of the indexer-side positioning element or the carousel-side positioning element comprises one or more magnets or electromagnets, and the other of the indexer-side positioning element and the carousel-side positioning element includes one or more ferrous surfaces configured to attract the one or more magnets or electromagnets.

8. The apparatus of claim 1, wherein the carousel-side positioning element comprises a carousel-side plate configured to be attached to a base of the carousel, the carousel-side plate comprising an opening configured to receive a spindle of the indexer-side positioning element.

9. The apparatus of claim 8, wherein the carousel-side plate comprises one or more ball bearings sized positioned to be received in corresponding grooves in the indexer-side positioning element to position the indexer-side positioning element with respect to the carousel-side positioning element.

10. The apparatus of claim 1, further comprising one or more fasteners configured to secure:
the indexer-side positioning element to the indexer;
the carousel-side positioning element to the carousel; or
the indexer-side positioning element to the carousel through the carousel-side positioning element.

11. An indexer for use with a microbial detection and/or enumeration system comprising a carousel configured to hold one or more cassettes and including an carousel-side positioning element configured to assist the indexer in rotating the carousel into predefined positions, the indexer comprising:
an indexer body housing a motor;
an indexer-side plate configured to be rotated by the motor; and
a spindle configured to receive the carousel-side positioning element, the spindle comprising one or more grooves configured to position the carousel with respect to the indexer as the carousel-side plate is lowered onto the spindle.

12. A carousel for use with a microbial detection and/or enumeration system comprising an indexer configured to rotate the carousel into predefined positions, the carousel comprising:
one or more cassette columns;
a carousel base supporting the one or more cassette columns; and
a carousel-side positioning element configured to position the carousel with respect to the indexer, the carousel-side positioning element comprising an opening configured to receive a spindle of the indexer, the opening including one or more protrusions corresponding to grooves in the spindle.

13. A method of manufacturing a system for positioning a carousel with respect to an indexer configured to rotate the carousel into predefined positions, the method comprising:
forming an indexer spindle on an indexer-side plate, the indexer spindle extending from a proximal end closest to the indexer-side plate to a distal end and tapering from a relatively large diameter at the proximal end to a relatively small diameter at the distal end;
forming one or more grooves in the indexer spindle;
accessing a carousel-side plate; and
forming an opening in the carousel-side plate, the forming comprising shaping one or more protrusions in the opening to correspond to the one or more grooves of the indexer spindle.

14. The method of claim 13, further comprising adding magnets or electromagnets to one or more of the indexer-side plate or the carousel-side plate.

15. The method of claim 13, further comprising securing one or more ball bearings in the indexer-side plate and forming one or more grooves in the carousel-side plate configured to receive the ball bearings to guide the carousel-side plate onto the indexer-side plate.

16. The method of claim 13, further comprising securing one or more ball bearings in the carousel-side plate and forming one or more grooves in the indexer-side plate configured to receive the ball bearings and to guide the carousel-side plate onto the indexer-side plate.

17. The method of claim 13, further comprising:
fixing the carousel-side plate to the carousel using one or more carousel-side fasteners; and
fixing the indexer-side plate to the indexer using one or more indexer-side fasteners.

18. A method of using a system for positioning a carousel with respect to an indexer configured to rotate the carousel into predefined positions, the method comprising:
attaching a carousel-side positioning element to the carousel, the carousel-side positioning element comprising an opening;
attaching an indexer-side positioning element to the indexer, the indexer-side positioning element comprising a spindle configured to be received in the opening of the carousel-side positioning element; and
dropping the carousel-side positioning element onto the indexer-side positioning element, wherein dropping the carousel-side positioning element causes one or more protrusions in the opening of the carousel-side positioning element to be received in one or more corresponding grooves on the spindle to automatically align the carousel with the indexer.

19. The method of claim 18, further comprising aligning one or more magnets or electromagnets on the carousel-side positioning element to one or more corresponding magnets or electromagnets on the indexer-side positioning element to secure the carousel to the indexer.

20. The method of claim 18, further comprising aligning one or more ball bearings on one of the carousel-side positioning element or the indexer-side positioning element to one or more grooves of the other of the carousel-side positioning element or the indexer-side positioning element.
